(19) United States
(12) Patent Application Publication
(10) Pub. No.: US 2021/0321886 A1
(43) Pub. Date: Oct. 21, 2021

(54) PORTABLE MONITORING APPARATUS, MONITORING DEVICE, MONITORING SYSTEM AND PATIENT STATUS MONITORING METHOD

(71) Applicant: SHENZHEN MINDRAY BIO-MEDICAL ELECTRONICS CO., LTD., Shenzhen (CN)

(72) Inventors: Bingbing XUE, Shenzhen (CN); Jianhui ZHANG, Shenzhen (CN); Qiling LIU, Shenzhen (CN); Xia JIANG, Shenzhen (CN); Jian REN, Shenzhen (CN); Jianfang CAO, Shenzhen (CN); Yu CHEN, Shenzhen (CN); Jian CEN, Shenzhen (CN); Zhonghua LIU, Shenzhen (CN); Kun JIAO, Shenzhen (CN)

(73) Assignee: SHENZHEN MINDRAY BIO-MEDICAL ELECTRONICS CO., LTD., Shenzhen (CN)

(21) Appl. No.: 17/362,851

(22) Filed: Jun. 29, 2021

Related U.S. Application Data
(63) Continuation-in-part of application No. PCT/CN2018/125662, filed on Dec. 29, 2018.
Application No. PCT/CN2018/125662 is a continuation-in-part of application No. PCT/CN2018/125795, filed on Dec. 29, 2018.

(51) Int. Cl. A61B 5/0205; A61B 5/00

(52) U.S. Cl. CPC A61B 5/02055; A61B 5/4806; A61B 5/7285; A61B 5/742; A61B 5/002; A61B 5/4824

(57) ABSTRACT

A portable monitoring apparatus, a monitoring device, and a monitoring system are disclosed. The portable monitoring apparatus includes a first type of sensor configured to acquire a physiological parameter value, which includes at least one of an electrocardiogram (ECG) parameter value, a respiratory parameter value, a blood oxygen parameter value, a blood pressure parameter value, and a body temperature parameter value; a second type of sensor configured to acquire a non-physiological parameter value, which includes at least one of a sleep parameter value, a motion parameter value, and a pain parameter value; and a processor configured to take the acquired physiological parameter value and non-physiological parameter value as patient status recovery parameter values and control to output the patient status recovery parameter values.

### CROSS-REFERENCE TO RELATED APPLICATIONS

This application is a continuation-in-part of PCT Application NO. PCT/CN2018/125662, and a continuation-in-part of PCT Application NO. PCT/CN2018/125795, both file Dec. 29, 2018, both of which are incorporated herein by reference in their entireties.

### TECHNICAL FIELD

This application relates to the technical field of patient status monitoring, in particular to a portable monitoring apparatus, monitoring device, monitoring system and patient status monitoring method.

### BACKGROUND

A traditional in-hospital monitoring device usually includes a bedside monitor for severe patients, which is arranged at the bedside of the patients and used to monitor status of the patients, enables doctors to learn about physical recovery state of the patients, and prompt possible physiological risks for the patients, and thus plays an important role in recovery and accident prevention for patients. The existing monitors are all ward-level monitors/bedside monitors placed at the bedside in a ward, which are typically immobile. For some patients, such as sub-severe patients, there is a need for real-time status monitoring and also a need for movement within a certain range, for example, taking a walk outside the ward. Therefore, how to meet the need to monitor status of sub-severe patients during their activity outside the ward has become a problem to be solved.

### SUMMARY

The disclosure provides a portable monitoring apparatus, a monitoring device, a monitoring system and a patient status monitoring method to solve the above problems.

An embodiment of the disclosure provides a portable monitoring apparatus, which includes a first type of sensor, a second type of sensor, and a processor. The first type of sensor is configured to acquire a physiological parameter value, which includes at least one of an electrocardiogram (ECG) parameter value, a respiratory parameter value, a blood oxygen parameter value, a blood pressure parameter value, and a body temperature parameter value. The second type of sensor is configured to acquire a non-physiological parameter value, which includes at least one of a sleep parameter value, a motion parameter value, and a pain parameter value. The processor is configured to take the acquired physiological parameter value and non-physiological parameter value as patient status recovery parameter values and control to output the patient status recovery parameter values.

An embodiment of the disclosure also provides a monitoring device, which includes a communication unit, a display screen, and a processor. The processor is configured to receive relevant data of patient status recovery parameter values through the communication unit and control the display screen to display the received relevant data of the patient status recovery parameter values, wherein the relevant data of the patient status recovery parameter values include a physiological parameter value and a non-physiological parameter value, the physiological parameter value includes at least one of an ECG parameter value, a respiratory parameter value, a blood oxygen parameter value, a blood pressure parameter value, and a body temperature parameter value, and the non-physiological parameter value includes at least one of a sleep parameter value, a motion parameter value, and a pain parameter value.

An embodiment of the disclosure also provides a monitoring system including a portable monitoring apparatus and a monitoring device, wherein the portable monitoring apparatus includes a first type of sensor, a second type of sensor, and a processor. The first type of sensor is configured to acquire a physiological parameter value, which includes at least one of an ECG parameter value, a respiratory parameter value, a blood oxygen parameter value, a blood pressure parameter value, and a body temperature parameter value. The second type of sensor is configured to acquire a non-physiological parameter value, which includes at least one of a sleep parameter value, a motion parameter value, and a pain parameter value. The processor is configured to take the acquired physiological parameter value and non-physiological parameter value as patient status recovery parameter values and control to output the patient status recovery parameter values. The monitoring device includes a communication unit, a display screen, and a processor. The processor is configured to receive relevant data of patient status recovery parameter values through the communication unit and control the display screen to display the received relevant data of the patient status recovery parameter values.

An embodiment of the disclosure also provides a patient status monitoring method, which is applied to a portable monitoring apparatus, the method including: acquiring, by a first type of sensor of the portable monitoring apparatus, a physiological parameter value, which includes at least one of an ECG parameter value, a respiratory parameter value, a blood oxygen parameter value, a blood pressure parameter value, and a body temperature parameter value; acquiring, by a second type of sensor of the portable monitoring apparatus, a non-physiological parameter value, which includes at least one of a sleep parameter value, a motion parameter value, and a pain parameter value; and taking the acquired physiological parameter value and non-physiological parameter value as patient status recovery parameter values, and outputting the patient status recovery parameter values.

An embodiment of the disclosure also provides a patient status monitoring method, which is applied to a monitoring device, the monitoring device includes a communication unit and a display screen, and the method includes: receiving relevant data of patient status recovery parameter values through the communication unit; and controlling the display screen to display the received relevant data of the patient status recovery parameter values, wherein the patient status recovery parameter values include a physiological parameter value and a non-physiological parameter value, the physiological parameter value includes at least one of an ECG parameter value, a respiratory parameter value, a blood oxygen parameter value, a blood pressure parameter value, and a body temperature parameter value, and the non-physiological parameter value includes at least one of a sleep parameter value, a motion parameter value, and a pain parameter value.

An embodiment of the disclosure also provides a wearable piece including a main body, the main body including a main body housing, and a processor and a first motion sensor arranged in the main body housing, wherein the processor is electrically connected to the first motion sensor, and the processor acquires a motion parameter value of a patient wearing the wearable piece according to a motion sensing signal generated by the first motion sensor and deduces an motion amount and/or sleep status of the patient wearing the wearable piece from the motion parameter value.

An embodiment of the disclosure also provides a portable monitoring apparatus including an ECG/respiratory lead cable, an ECG module, and at least three electrode pad connectors, wherein one end of the ECG/respiratory lead cable is configured to be connected to a wearable piece; the ECG/respiratory lead cable is serially provided, from one end close to the wearable piece to the other end away from the wearable piece, with the ECG module and the at least three electrode pad connectors in sequence; and the electrode pad connectors are configured to clamp electrode pads.

### BRIEF DESCRIPTION OF THE DRAWINGS

In order to illustrate the technical solutions in the embodiments of the disclosure more clearly, a brief introduction to the drawings required for the embodiments will be provided below. Obviously, the drawings in the following description are merely some of the embodiments of the disclosure, and those of ordinary skill in the art would also obtain other drawings according to these drawings without involving any inventive effort.

FIG. 1 is a schematic diagram of a monitoring system used in a hospital according to an embodiment of the disclosure;

FIG. 2 is a block diagram of a portable monitoring apparatus according to an embodiment of the disclosure;

FIG. 3 is a schematic structure diagram of a portable monitoring apparatus according to an embodiment of the disclosure;

FIG. 4 is a schematic diagram of a parameter interface displayed on a display screen of a portable monitoring apparatus according to an embodiment of the disclosure;

FIG. 5 is a schematic diagram of a pairing prompt message displayed by a portable monitoring apparatus according to an embodiment of the disclosure;

FIG. 6 is a schematic diagram of pairing displayed by a portable monitoring apparatus according to an embodiment of the disclosure;

FIG. 7 is a block diagram of a monitoring device according to an embodiment of the disclosure;

FIG. 8 is a schematic diagram of a parameter interface displayed by a monitoring device according to an embodiment of the disclosure;

FIG. 9 is a schematic diagram of a pairing prompt message displayed by a monitoring device according to an embodiment of the disclosure;

FIG. 10 is a schematic diagram of pairing displayed by a monitoring device according to an embodiment of the disclosure;

FIG. 11 is a schematic diagram of a parameter interface displayed by a monitoring device according to an embodiment of the disclosure;

FIG. 12 is a system frame diagram of a multi-parameter monitor or module assembly according to an embodiment of the disclosure;

FIG. 13 is a flow chart of a patient status monitoring method according to an embodiment of the disclosure;

FIG. 14 is a flow chart of a patient status monitoring method according to an embodiment of the disclosure;

FIG. 15 is a flow chart of a patient status monitoring method according to an embodiment of the disclosure; and

FIG. 16 is a schematic diagram of an interface displayed on a display screen of a portable monitoring apparatus according to another embodiment of the disclosure.

### DETAILED DESCRIPTIONS

The description has been made with reference to various exemplary embodiments herein. However, those skilled in the art would have appreciated that changes and modifications could have been made to the exemplary embodiments without departing from the scope herein. For example, various operation steps and assemblies for executing operation steps may be implemented in different ways according to a specific application or considering any number of cost functions associated with the operation of the system (for example, one or more steps may be deleted, modified or incorporated into other steps).

The terms “first”, “second” and the like in the specification and the claims of the disclosure and the above drawings are used to distinguish different objects, but not to describe a specific order. In addition, the terms “comprise”, “include”, “have”, and any variations thereof are intended to cover non-exclusive inclusion. For example, a process, a method, a system, a product, or a device that includes a series of steps or units is not limited to the listed steps or units, but optionally further includes unlisted steps or units, or optionally further includes other steps or units inherent in these processes, methods, or devices.

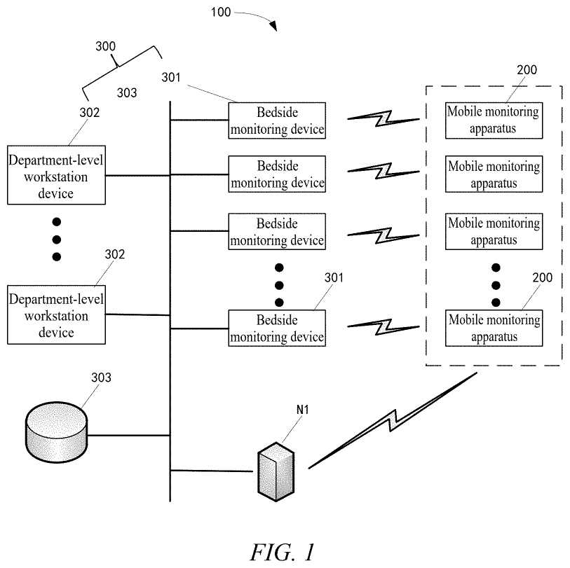

Referring to FIG. 1, provided is a schematic diagram of a monitoring system 100 used in hospital. By using the monitoring system 100, the data of monitors can be saved as a whole, patient information and nursing information can be managed centrally and can be stored in association, which facilitates the storage of historical data and associated alarming. In the monitoring system 100 shown in FIG. 1, the monitoring system includes at least one portable monitoring apparatus 200 and at least one monitoring device 300. The at least one monitoring device 300 includes at least one of a bedside monitoring device, a department-level workstation device and a hospital-level data center/hospital-level emergency center management device. The portable monitoring apparatus 200 can be a wearable monitoring device.

As shown in FIG. 1, the monitoring device 300 includes bedside monitors 301, where each ward bed can be provided with one of the bedside monitors 301. The bedside monitor 301 can be a multi-parameter monitor or a plug-in monitor. In addition, each bedside monitor 301 can also be paired with one portable monitoring apparatus 200 for data transmission. The portable monitoring apparatus 200 provides a simple and mobile multi-parameter monitor or module assembly, which may be worn by the patient for mobile monitoring. After the portable monitoring apparatus 200 communicates with the bedside monitor 301 by wire or wirelessly, the patient status data generated by mobile monitoring can be transmitted to the bedside monitor 301 for display. As shown in FIG. 1, the monitoring device 300 may also include a department-level workstation device 302 and a hospital-level data center/hospital-level emergency center management device 303. The patient status data generated by the portable monitoring apparatus 200 is transmitted to the department-level workstation device 302 for doctors or nurses to check, or transmitted through the bedside monitors 301 to the hospital-level data center/hospital-level emergency center management device 303 for storage and/or display.

In addition, the portable monitoring apparatus 200 can also directly transmit the patient status data generated by mobile monitoring through a wireless network node N1 provided in the hospital to the department-level workstation device 302 for storage and display, or transmit the patient status data generated by mobile monitoring through the wireless network node N1 provided in the hospital to the hospital-level data center/hospital-level emergency center management device 303 for storage. It can be seen that the data corresponding to the patient status parameter values displayed on the bedside monitor 301 can be derived from sensor accessories directly connected to the bedside monitor, or from the portable monitoring apparatus 200, or from the department-level workstation device 302 and the hospital-level data center/hospital-level emergency center management device 303.

Here, each portable monitoring apparatus 200 can also store the patient status data acquired by itself, and the bedside monitor 301 can also store the patient status data acquired by the sensor accessories connected to the bedside monitor and store the patient status data received from the portable monitoring apparatus 200, department-level workstation device 302, hospital-level data center/hospital-level emergency center management device 303, etc. The department-level workstation device 302 and the hospital-level data center/hospital-level emergency center management device 303 can store patient status data sent by any portable monitoring apparatus 200.

Referring to FIG. 2, provided is a block diagram of the portable monitoring apparatus 200. As shown in FIG. 2, the portable monitoring apparatus 200 includes a first type of sensor 21, a second type of sensor 22, and a processor 23. The processor 23 is connected to the first type of sensor 21 and the second type of sensor 22.

The first type of sensor 21 is configured to acquire a physiological parameter value, which includes at least one of an electrocardiogram (ECG) parameter value, a respiratory parameter value, a blood oxygen parameter value, a blood pressure parameter value, and a body temperature parameter value.

The second type of sensor 22 is configured to acquire a non-physiological parameter value, which includes at least one of a sleep parameter value, a motion parameter value, and a pain parameter value.

The processor 23 is configured to take the acquired physiological parameter value and non-physiological parameter value as patient status recovery parameter values and control to output the patient status recovery parameter values.

Here, the processor 23 summarizes the acquired physiological parameter value and non-physiological parameter value to form patient status recovery parameter values.

Therefore, in this application, the portable monitoring apparatus 200 may obtain the physiological parameter value and the non-physiological parameter value at the same time, and may obtain physiological status such as ECG and respiration as well as non-physiological status such as sleep and motion states of the user, and thus may detect the status of the patient more comprehensively.

In one embodiment, the second type of sensor 22 is a motion sensor. The motion sensor is configured to sense an acceleration of the wearable piece 201 after being worn by the user. The processor 23 is electrically connected to the motion sensor and acquires a motion parameter value of a patient wearing the wearable piece 201 according to a motion sensing signal generated by the motion sensor and deduces an motion amount and/or sleep status of the patient wearing the wearable piece 201 from the motion parameter value.

In one embodiment, the motion sensor may record the patient's motion parameter value in real time. Therefore, the processor 23 may deduce an amount of motion and/or sleep status of the patient from the motion parameter value obtained by the motion sensor for the implementation of enhanced recovery after surgery (ERAS), which contributes to rapid recovery of the patient. Meanwhile, data processing can be carried out in combination with the motion parameter value obtained by the motion sensor during the process of calculation and analysis of the ECG parameter value, respiratory parameter value, blood oxygen parameter value, blood pressure parameter value and body temperature parameter value by the processor 23, so as to eliminate the interference with the ECG parameter value, respiratory parameter value, blood oxygen parameter value, blood pressure parameter value and body temperature parameter value due to movement of the patient wearing the wearable piece 201, thereby providing more accurate analysis results. Here, the portable monitoring apparatus 200 may be a wearable monitoring apparatus, which is configured to be worn by a patient for activities outside the ward, and at the same time can continuously monitor the patient status recovery parameter value, thus meeting the need to monitor status of the patient (for example, a sub-severe patient) during his/her activities outside the ward.

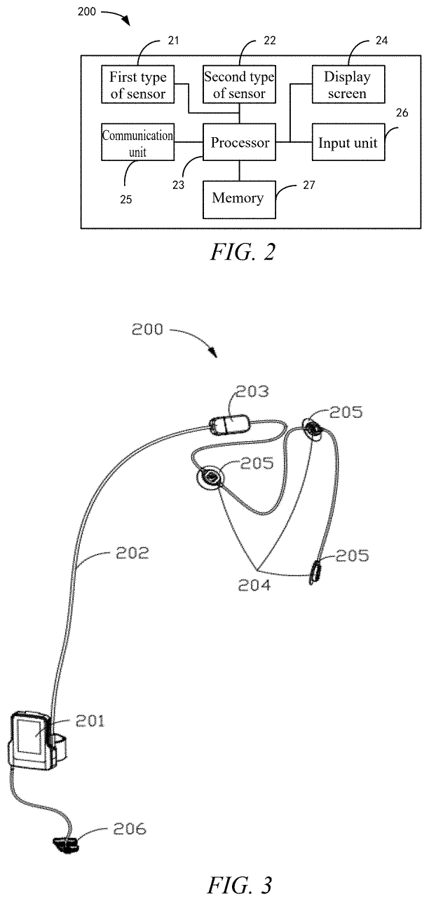

Referring also to FIG. 3, provided is a schematic structure diagram of the portable monitoring apparatus 200. The portable monitoring apparatus 200 further includes the wearable piece 201, a parameter measurement cable 202, an ECG module 203, and several electrode pad connectors 204. The wearable piece 201 is connected to one end of the parameter measurement cable 202. The parameter measurement cable 202 is serially provided, from one end close to the wearable piece 201 to the other end away from the wearable piece 201, with the ECG module 203 and the several electrode pad connectors 204 in sequence. The electrode pad connectors 204 are configured to clamp electrode pads 205. Still further, in some modified embodiments, the parameter measurement cable 202 may be a one-cord parameter measurement cable composed of one cable structure serially provided with the ECG module 203 and the plurality of electrode pad connectors 204 in sequence, or may alternatively consist of a furcate cable structure. If the parameter measurement cable 202 is of a furcate cable structure, the parameter measurement cable 202 includes a trunk and a plurality of branches. One end of the trunk is connected to the wearable piece 201, and the other end of the trunk is connected to the plurality of branches. Each of the branches is provided with at least one electrode pad connector, and the ECG module 203 is arranged at any position on the trunk. It should be understood that in an embodiment of the disclosure, the ECG module 203 can be configured as a separate wearable piece, which can communicate with the wearable piece 201 for data transmission, or otherwise, instead of communicating with the wearable piece 201, can independently send the patient status data collected by itself to the department-level workstation device 302, the hospital-level data center/hospital-level emergency center management device 303, etc.

Specifically, the wearable piece 201 is configured to be tied to a wrist of the patient to monitor the physiological parameter value and/or non-physiological parameter value of the patient. Each electrode pad connector 204 is used to clamp one electrode pad 205, and each electrode pad 205 is configured to be attached to a certain part of the patient's body to measure the physiological parameter value and/or non-physiological parameter value or impedance signal of the part. A defibrillator protection circuit is provided in the ECG module 203, and the defibrillator protection circuit is used as a protection circuit to prevent the portable monitoring apparatus 200 from being damaged when defibrillating the patient's heart to restore a normal heartbeat when necessary. In this application, the ECG module 203 and the wearable piece 201 are provided separately, which makes the wearable piece 201 more compact and mobile, and prevents interference with signals in the wearable piece 201 from strong currents applied to the ECG module 203. In this embodiment, the wearable piece 201 can be worn on a wrist of the patient, and the ECG/respiratory monitoring function is realized by using a one-cord ECG/respiratory lead cable 30. The whole portable monitoring apparatus 200 is small, light and comfortable to wear, which minimizes the influence of the portable monitoring apparatus 200 on the daily life of the patient. In some modified embodiments, the ECG/respiratory lead cable 30 may be a one-cord ECG/respiratory lead cable composed of one cable structure serially provided with the ECG module 203 and the at least three electrode pad connectors 204 in sequence, or may alternatively consist of a furcate cable structure. If the ECG/respiratory lead cable 30 if of a furcate cable structure, the ECG/respiratory lead cable 30 includes a trunk and at least three branches. One end of the trunk is connected to the wearable piece 201, and the other end of the trunk is connected to at least three branches. Each of the branches is provided with at least one electrode pad connector 204, and the ECG module 203 is arranged at any position on the trunk. Each electrode pad connector 204 is configured to clamp one electrode pad 205, and each electrode pad 205 is configured to be attached to a certain part of the patient's body to measure the physiological data signal or impedance signal of the part. The ECG module 203 contains a defibrillator protection circuit, which is used as a protection circuit to prevent the ECG detection system from being damaged when defibrillating the patient's heart to restore a normal heartbeat when necessary. In the disclosure, the ECG module 203 and the wearable piece 201 are provided separately, which makes the wearable piece 201 more compact and mobile, and prevents interference with signals in the wearable piece 201 from strong currents applied to the ECG module 203.

In some embodiments, a motion sensor may be provided, which may be located in the wearable piece 201 or the ECG module 203. In another embodiment, motion sensors may be provided in the wearable piece 201 and the ECG module 203, respectively. As the ECG module 203 is clamped onto a collar of the patient, the motion sensor in the ECG module 203 can measure the patient's motion parameter value more accurately without interference caused by the patient's arm motion, thus accurately deducing an motion amount and/or sleep status of the patient.

Here, in this application, the first type of sensor 21 includes at least one of an ECG sensor, a respiration sensor, a blood oxygen sensor, a blood pressure sensor, and a temperature sensor. In some embodiments, the first type of sensor 21 may include the ECG sensor, respiration sensor, blood oxygen sensor, blood pressure sensor, and temperature sensor at the same time, and acquire and monitor the ECG parameter value, respiration parameter value, blood oxygen parameter value, blood pressure parameter value, and body temperature parameter value simultaneously.

At least some electrode pads 205 of the electrode pads 205 clamped by the electrode pad connectors 204 constitute the ECG sensor, wherein there are a plurality of ECG sensors, and each electrode pad 205 corresponds to one ECG sensor. By attaching the electrode pads 205 constituting the ECG sensors to corresponding parts of the patient's body, the ECG parameter value can be acquired and the corresponding ECG data can be obtained.

Here, the respiration sensor is configured to monitor the respiratory parameter value, such as respiratory rate. The respiration sensor may also include at least one type of electrode pads 205 clamped by a plurality of electrode pad connectors 204. In some embodiments, the respiration sensor and the ECG sensor are integrated and share common electrode pads 205.

As shown in FIG. 3, the blood oxygen (SPO2) sensor includes a blood oxygen probe 206 electrically connected to the wearable piece 201, and the blood oxygen probe 206 can be of a clamping structure, which is configured to clamp a finger of the patient and measure the blood oxygen parameter value, such as a blood oxygen concentration, through light intensity signals.

The blood pressure sensor can be disposed on the wearable piece 201, for example, on the back of the wearable piece 201, for monitoring the blood pressure parameter value, such as the high pressure value and low pressure value.

In some embodiments, the blood pressure sensor can be arranged in a strap structure, which is tied to an arm of the patient, and the blood pressure sensor is connected to the wearable piece 201 by wire or wirelessly and sends the monitored blood pressure parameter value to the wearable piece 201. The arm onto which the blood pressure sensor is tied is different from the arm that wears the wearable piece 201, so as to avoid interference.

The temperature sensor can also be integrated with the ECG sensor, that is, shares the same electrode pads 205. By attaching the electrode pads 205 to corresponding parts of the patient's body, the acquisition/monitoring of the ECG parameter value and temperature parameter value can be realized at the same time.

In some embodiments, the temperature sensor may be alternatively arranged in the ECG module 203 and include a body temperature probe extending from the ECG module 203. Because the ECG module 203 is located close to an armpit of the user, the cable length of the body temperature probe can be shortened, which is convenient for measuring the armpit temperature of the patient and further improves wearing comfort.

Here, the ECG module 203 includes an ECG processing circuit for processing the ECG parameter value acquired by the ECG sensor and send the processed ECG parameter value to the wearable piece 201. The ECG module 203 may also include measurement circuits such as a blood pressure measurement processing circuit, a blood oxygen measurement processing circuit, a temperature measurement processing circuit, etc., which are configured to process the parameter values acquired by respective sensors and then send them to the wearable piece 201.

Here, the second type of sensor 22 includes at least one of a motion sensor and a pain sensor. In this embodiment, the second type of sensor 22 includes a motion sensor and a pain sensor.

The motion sensor is configured to obtain acceleration information indicating sleep parameter value and motion parameter value of the patient, that is, the motion parameter value and sleep parameter value of the patient can be derived from the acceleration value monitored by the motion sensor 22. For example, when the acceleration value is zero, it can be determined that the patient is still, and when the acceleration value changes, it indicates that the patient is moving, and the change frequency of the acceleration value indicates the motion parameter value such as pace frequency and speed of the patient's movement, while the statistics of the time when the acceleration value is zero may indicate the sleep parameter value such as sleep time.

There may be a plurality of motion sensors, which are respectively arranged in the wearable piece 201 and the ECG module 203, which can effectively reduce false motion detection caused by movements of the arm and improve the statistical accuracy of the patient's motion time and sleep time.

Here, the pain sensor may include at least one of a humidity sensor, a sound sensor, etc. Patients may sweat when they are in pain, and the more pain they feel, the more they sweat, so the humidity value detected by the humidity sensor can also indicate the pain parameter value, that is, the pain level. For another example, when patients are in pain, they usually cries because they cannot bear it. When the voice content of the user is detected by a sound sensor and determined to be a pain groan, the pain parameter value, that is, the pain level, can be indicated according to the volume.

Here, the processor 23 is disposed in the wearable piece 201. In some embodiments, the processor 23 obtains the motion parameter value and sleep parameter value of the patient by receiving the acceleration value monitored by the motion sensor, and obtains the pain parameter value of the patient by receiving the humidity value monitored by the humidity sensor and/or the voice message by the sound sensor. Therefore, the non-physiological parameter value monitored/acquired by the second type of sensor 22 including at least one of the sleep parameter value, motion parameter value and pain parameter value refers to the parameter value monitored/acquired by the second type of sensor 22 which indirectly indicates at least one of the sleep parameter value, motion parameter value and pain parameter value.

As shown in FIG. 2, the portable monitoring apparatus 200 further includes a display screen 24, and the processor 23 is connected to the display screen 24 for controlling the display of the patient status recovery parameter values on the display screen 24. That is, the processor 23 controlling the output of the patient status recovery parameter values is to control the display of the patient status recovery parameter values on the display screen 24.

Here, the display screen 24 is disposed on the wearable piece 201, specifically, the display screen 24 is disposed on an outer surface of the wearable piece 201.

As shown in FIG. 3, the wearable piece 201 is a wristband device to be worn on a wrist of the patient. When the wearable piece 201 is worn on the wrist of the patient, the display screen 24 is located on the side facing away from the wrist of the patient.

In some embodiments, the back of the wearable piece 201 is also integrated with a heart rate sensor for measuring the heart rate/pulse rate after the wearable piece 201 is worn on the wrist, wherein the heart rate sensor is a photoelectric transducer. The heart rate/pulse rate measured by the heart rate sensor can be used as the ECG parameter value alone or combined with the ECG parameter value measured by the ECG sensor as the final ECG parameter value.

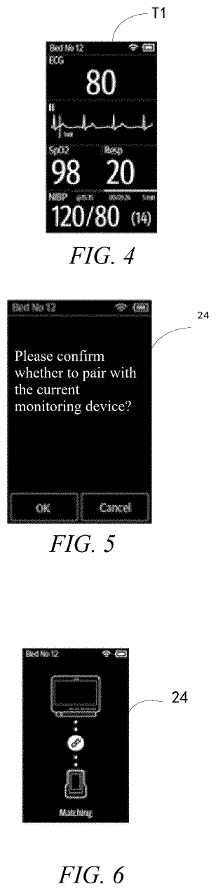

Referring to FIG. 4, provided is a schematic diagram of a parameter interface displayed on the display screen 24 of the portable monitoring apparatus 200. Further, the processor 23 controls the display screen 24 to display a parameter interface T1 including at least one of the patient status recovery parameter values when controlling to display the patient status recovery parameter values on the display screen 24. For example, as shown in FIG. 4, the processor 23 controls the display screen 24 to display the parameter interface T1 including the ECG parameter value, blood oxygen parameter value, respiratory parameter value and blood pressure parameter value, where the ECG parameter value is shown as an ECG curve, and the blood oxygen parameter value is 98, the respiratory rate is 20, and the blood pressure is 120/80. Or, in some embodiments, the processor 23 controls the display screen 24 to display a parameter interface including only the blood pressure parameter value.

In some embodiments, the processor 23 is further configured to control to switch the parameter value displayed in the parameter interface T1 in response to an operation of the user or according to a preset time interval.

That is, when the patient status recovery parameter values include several parameter values, and only some of the parameter values are displayed in the parameter interface T1 at one time, the parameter values displayed in the parameter interface can be controlled to switch in response to an operation by the user or according to a preset time interval, for example, switching the blood pressure parameter value currently displayed in the parameter interface to the body temperature parameter value.

In some embodiments, the display screen 24 is a touch screen, and the processor 23 controls to switch the parameter values displayed in the parameter interface in response to a sliding touch operation inputted by the user on the display screen 24. In some embodiments, the portable monitoring apparatus 200 includes a mechanical switch key, and the processor 23 controls to switch the parameter values displayed in the parameter interface in response to the depressing of the mechanical switch key.

In some embodiments, when the patient status recovery parameter value displayed on the display screen 24 is the motion parameter value, the processor 23 also controls to display sub-motion parameter values included in the motion parameter value in a ring-shaped form, and the sub-motion parameter value includes at least one of a number of steps, a step frequency, a distance of motion, calories, a time of motion and a route of motion.

For example, when the processor 23 switches the parameter value displayed in the parameter interface T1 to the motion parameter value, a plurality of sub-motion parameter values included in the motion parameter value are displayed in the parameter interface at the same time, and the plurality of sub-motion parameter values are arranged in a ring shape.

As shown in FIG. 2, the portable monitoring apparatus 200 further includes a communication unit 25. The processor 23 is connected to the communication unit 25, and is further configured to establish a communication connection between the portable monitoring apparatus 200 and a target monitoring device 300 through the communication unit 25, and to send the patient status recovery parameter values to the target monitoring device 300 through the communication unit 25, and the patient status recovery parameter values are outputted through the target monitoring device 300. Here, the target monitoring device 300 includes at least one of a bedside monitoring device 301, a department-level workstation device 302, and a hospital-level data center/hospital-level emergency center management device 303. In some embodiments, the communication connection between the portable monitoring apparatus 200 and the target monitoring device 300 is a wireless communication connection.

Here, the patient status recovery parameter values can be displayed by the target monitoring device 300 and can be further stored in the target monitoring device 300.

The communication unit 25 is provided in the wearable piece 201.

In some embodiments, the communication unit 25 includes a Bluetooth module, and the portable monitoring apparatus 200 communicates with other portable monitoring apparatus 200 through the Bluetooth module. The wearable piece 201 is used as the main communication device and is responsible for data transmission with the target monitoring device 300. The wearable piece 201 obtains the patient status recovery parameter values from other portable monitoring apparatus 200 through the Bluetooth module and sends them to the target monitoring device 300.

In a variant, the wearable piece 201 of the portable monitoring apparatus 200 only includes the communication unit 25 and the display screen 24, and the communication unit 25 includes a wireless communication module as a main communication device. The first type of sensor 21 and the second type of sensor 22 are both patch detection devices. A plurality of the patch detection devices also have near field communication modules such as Bluetooth modules and NFC modules and send the acquired physiological parameter value and non-physiological parameter value to the wearable piece 201 for display on the display screen 24. In addition, the acquired patient recovery state data including the physiological parameter values and non-physiological parameter values can also be sent to the target monitoring device 300 through the communication unit 25 of the wearable piece 201.

In some embodiments, when the processor 23 sends the patient status recovery parameter values to the target monitoring device 300 through the communication unit, it also sends display mode information to the target monitoring device 300, such that the target monitoring device 300 displays the patient status recovery parameter values according to the display mode information.

Here, the display mode information defines display positions of relevant data of the physiological parameter value and non-physiological parameter value in the parameter interface for displaying the patient status recovery parameter values, wherein the relevant data of the physiological parameter value is displayed in a first area of the parameter interface and the relevant data of the non-physiological parameter value is displayed in a second area of the parameter interface.

Therefore, when the target monitoring device 300 receives the patient status recovery parameter values, it will display the parameter interface according to the display mode information, and display the relevant data of physiological parameter value in the first area of the parameter interface, and the relevant data of non-physiological parameter value in the second area of the parameter interface.

Here, the relevant data of the physiological parameter value includes real-time data of the physiological parameter value, and the relevant data of the non-physiological parameter value includes real-time data of the non-physiological parameter value. That is, after the portable monitoring apparatus 200 sends the data of physiological parameter value and non-physiological parameter value acquired in real time to the target monitoring device 300, the data of the physiological parameter value and the data of the non-physiological parameter values acquired in real time will be displayed in different areas of the parameter interface.

Further, the relevant data of the non-physiological parameter value further includes trend data of the non-physiological parameter value. Here, the trend data of the non-physiological parameter value includes multiple historical data of the non-physiological parameter value monitored at different times arranged according to the monitoring time, showing a trend and forming the trend data.

Here, the trend data includes trend data of the plurality of non-physiological parameter values monitored within a preset time period, for example, trend data of the plurality of non-physiological parameter values monitored within the last week.

Here, since there are time intervals in the acquisition of the data of the physiological parameter value and non-physiological parameter value, the real-time data of the physiological parameter value and non-physiological parameter value may refer to the data of the physiological parameter value and non-physiological parameter value obtained in the latest measurement.

In some embodiments, when the target monitoring device 300 is the bedside monitor 301, the processor 23 also performs a pairing operation with the target monitoring device 300 while the communication unit 25 establishes the communication connection between the portable monitoring apparatus 200 and the target monitoring device 300 for the first time, and the communication connection between the portable monitoring apparatus 200 and the target monitoring device 300 is established after successful pairing with the target monitoring device 300.

Referring to FIG. 5, provided is a schematic diagram of a pairing prompt message displayed by the portable monitoring apparatus 200. As shown in FIG. 5, before performing the pairing operation with the target monitoring device, the processor 23 generates a pairing prompt message to prompt the user to choose whether to pair the portable monitoring apparatus 200 with the target monitoring device 300, and performs the pairing operation with the target monitoring device 300 if the user chooses “yes”. Here, the pairing prompt message can be a text message, where the processor 23 controls the display screen 24 to display the pairing prompt message in text form, such as “Please confirm whether to pair with the current monitoring device”.

Referring also to FIG. 6, provided is a schematic diagram of pairing displayed by the portable monitoring apparatus 200. When the user chooses “yes”, the processor 23 performs the pairing operation with the target monitoring device 300, and controls the display screen 24 to display a pairing indication message indicating a current pairing process, for example, the message as shown in FIG. 6 indicating that the portable monitoring apparatus 200 and the target monitoring device 300 are pairing.

Here, after the portable monitoring apparatus 200 is successfully paired with the target monitoring device 300, the processor 23 controls the portable monitoring apparatus 200 to restore the previously displayed parameter interface including the patient status recovery parameter values.

Here, the processor 23 is further configured to, after the portable monitoring apparatus 100 is successfully paired with the target monitoring device 200, control to synchronize real-time data and historical data of the patient status recovery parameter values detected by the portable monitoring apparatus 100 to the target monitoring device 200. Therefore, after the portable monitoring apparatus 200 is successfully paired with the target monitoring device 300, the target monitoring device 300 will also display the parameter interface including the patient status recovery parameter values. Therefore, the portable monitoring apparatus 200 and the target monitoring device display respective parameter interfaces at the same time.

Here, the parameter interface displayed by the portable monitoring apparatus 200 and the parameter interface displayed by the target monitoring device both show the relevant data of the patient status recovery parameter values based on ERAS.

The patient recovery guide defines the physiological and non-physiological parameter values of the patient status recovery parameter values to be detected during the patient recovery process. All or part of the parameter values of the patient status recovery parameter values defined in the patient recovery guide are selectively shown in the parameter interface displayed by the target monitoring device 300, and some of the parameter values of the patient status recovery parameter values defined in the patient recovery guide are selectively shown in the parameter interface displayed by the portable monitoring apparatus 200.

The type/number of patient status recovery parameter values in the parameter interface displayed by the portable monitoring apparatus 200 is less than the type/number of patient status recovery parameter values in the parameter interface displayed by the target monitoring device 300.

In some embodiments, the patient status recovery parameter values in the parameter interface displayed by the portable monitoring apparatus 200 are only some of the patient status recovery parameter values in the parameter interface displayed by the target monitoring device 300.

For example, as shown in FIG. 4, the parameter interface displayed by the portable monitoring apparatus 200 includes the ECG parameter value, blood oxygen parameter value, respiratory parameter value, and blood pressure parameter value, while the parameter interface displayed by the target monitoring device 300 may include the ECG parameter value, blood oxygen parameter value, respiratory parameter value, blood pressure parameter value, body temperature parameter value, motion parameter value, sleep parameter value, etc.

In some embodiments, the communication unit 25 includes a WIFI communication module and at least one of a Bluetooth module, a WMTS communication module and an NFC communication module. When the portable monitoring apparatus 100 is located in a ward, the portable monitoring apparatus 200 establishes a Bluetooth connection, a WMTS communication connection or an NFC communication connection with the bedside monitoring device 301 through the Bluetooth module, the WMTS communication module or the NFC communication module, and synchronizes real-time data and historical data of the patient status recovery parameter values detected by the portable monitoring apparatus 200 to the bedside monitoring device 301. Here, the real-time data and historical data of the patient status recovery parameter values can be further synchronized to the department-level workstation device 302 and/or the hospital-level data center/hospital-level emergency center management device 303 through the bedside monitoring device 301.

When the portable monitoring apparatus 200 is located outside the ward, the portable monitoring apparatus 200 establishes a WIFI communication connection with the department-level workstation device 302 and/or hospital-level data center/hospital-level emergency center management device 303 through a WIFI module. The real-time data and historical data of the patient status recovery parameter values detected by the portable monitoring apparatus 200 are synchronized to the department-level workstation device 302 and/or the hospital-level data center/hospital-level emergency center management device 303, and then the real-time data and historical data of the patient status recovery parameter values are further synchronized to the bedside monitoring device 302 through the department-level workstation device 302 and/or the hospital-level data center/hospital-level emergency center management device 303.

Therefore, whether the patient wearing the portable monitoring apparatus 200 is inside the ward or outside the ward, the relevant data of the patient status recovery parameter values monitored by the portable monitoring apparatus 200 will be synchronized to the bedside monitoring device 302, the department-level workstation device 302 and/or the hospital-level data center/hospital-level emergency center management device 303, so that the monitoring system 100 can save the data monitored by the portable monitoring apparatuses 200 as a whole, which is convenient for centralized management of patient information.

Because the power consumption of the Bluetooth module, WMTS communication module and NFC communication module is very low, when the portable monitoring apparatus 100 is located in the ward, the portable monitoring apparatus 200 establishes the Bluetooth connection, WMTS communication connection or NFC communication connection with the bedside monitoring device 301 through the Bluetooth module, WMTS communication module or NFC communication module, which can effectively reduce the power consumption and improve the battery endurance of the portable monitoring apparatus 100.

In some embodiments, the processor 23 is further configured to control the display screen 23 to display a lock screen interface when the portable monitoring apparatus 200 has received no user operation for a time longer than a preset time duration, and to unlock the portable monitoring apparatus 200 and resume the display of the parameter interface in response to any sliding operation by the user on the display screen.

The number of types of patient recovery parameter values included in the lock screen interface displayed by the portable monitoring apparatus 200 is less than the number of types of patient recovery parameter values in the parameter interface displayed after unlocking by the portable monitoring apparatus 200.

The lock screen interface can be a simplified interface of the unlocked parameter interface, where only some of the patient recovery parameter values displayed by the unlocked parameter interface are shown.

In some embodiments, the lock screen interface may also display only time information.

In some embodiments, the portable monitoring apparatus 200 further includes an input unit 26, and the processor 23 is further configured to set a current pain level of the user for the portable monitoring apparatus 200 in response to a pain level setting operation input through the input unit 26 to obtain the pain parameter value.

That is, in some embodiments, the pain parameter value may be a pain level value directly inputted by doctors, nurses or the patients themselves.

The input unit 26 can be a touch panel, which is integrated with the display screen 24 into a touch screen that enables input of the pain level value through an input box displayed on the display screen 24.

In some embodiments, the input unit 26 may also be a mechanical key, and doctors, nurses or the patients themselves can input the pain level value by operating the mechanical key.

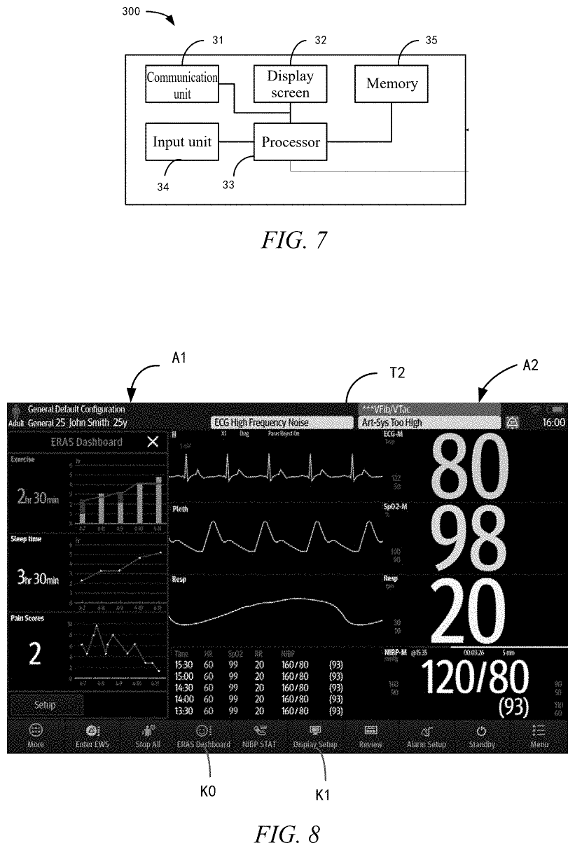

Referring to FIG. 7, provided is a block diagram of the monitoring device 300. As shown in FIG. 7, the monitoring device 300 includes a communication unit 31, a display screen 32, and a processor 33. The processor 33 is configured to receive relevant data of patient status recovery parameter values through the communication unit 31 and control the display screen 32 to display the received relevant data of the patient status recovery parameter values, wherein the relevant data of the patient status recovery parameter values include a physiological parameter value and a non-physiological parameter value, the physiological parameter value includes at least one of an ECG parameter value, a respiratory parameter value, a blood oxygen parameter value, a blood pressure parameter value, and a body temperature parameter value, and the non-physiological parameter value includes at least one of a sleep parameter value, a motion parameter value, and a pain parameter value.

Here, the communication unit 31 is configured to establish a communication connection with the portable monitoring apparatus 200, and the processor 33 receives the relevant data of the patient status recovery parameter values acquired by the portable monitoring apparatus 200 from the portable monitoring apparatus 200 through the communication unit 31.

Referring also to FIG. 8, provided is a schematic diagram of a parameter interface T2 displayed by the monitoring device 300. The processor 33 controls the display screen 32 to display the above-mentioned parameter interface T2 including the received relevant data of the patient status recovery parameter values, wherein the relevant data of the non-physiological parameter value is displayed in a first area A1 of the parameter interface T2 and the relevant data of the physiological parameter value is displayed in a second area A2 of the parameter interface T2.

As shown in FIG. 8, the first area A1 of the parameter interface T2 is the left area of the parameter interface T2, and the second area A2 is the right area of the parameter interface T2. The sizes of the first area A1 and the second area A2 may be equal or unequal. In some embodiments, the size of the first area A1 is smaller than the size of the second area A2.

Here, the relevant data of the physiological parameter value includes at least real-time data of the physiological parameter value, and the relevant data of the non-physiological parameter value includes real-time data of the non-physiological parameter value.

Further, the relevant data of the non-physiological parameter value further includes trend data of the non-physiological parameter value. Here, the trend data of the non-physiological parameter value includes multiple historical data of the non-physiological parameter value monitored at different times arranged according to the monitoring time, showing a trend and forming the trend data.

Here, the trend data includes trend data of the plurality of non-physiological parameter values detected within a preset time period, for example, within the last week.

As shown in FIG. 8, the first area A1 of the parameter interface T2 shows an motion time (Motion), i.e. the motion parameter value, and historical trend data of the motion parameter value composed of a plurality of historical motion events located to the right of the motion time.

Here, the historical trend data of the motion parameter value further includes the target motion parameter value and the actual motion parameter value for each motion. For example, in FIG. 8, the black column is the target motion parameter value, while the gray part in the corresponding column is the actual motion parameter value. Therefore, through the historical trend data in FIG. 2, the information of the achievement rate of each motion time can also be illustrated.

The first area A1 also displays a sleep time, i.e. the sleep parameter value, and historical trend data of the sleep parameter value composed of a plurality of historical sleep parameter values located to the right of sleep time. The historical trend data of the sleep parameter value includes the sleep time of multiple sleeps.

The first area A1 also displays the pain parameter value, i.e., the pain level value, and the historical trend data of the pain parameter value composed of a plurality of historical pain parameter values located to the right of the pain parameter value. The historical trend data of pain parameter value includes pain level values obtained for multiple times.

Here, the motion parameter value, the sleep parameter value and the pain parameter value are sequentially arranged and shown from top to bottom.

Obviously, the arrangement order of the motion parameter value, sleep parameter value and pain parameter value in the parameter interface can also be in other alternative orders, for example, arranged and shown in a sequence of the sleep parameter value, motion parameter value and pain parameter value from top to bottom.

For example, the relevant data of the physiological parameter value displayed in the second area A1 includes the real-time value and waveform of the physiological parameter value.

For example, as shown in FIG. 8, the left part of the second area A1 displays the ECG curve, the blood oxygen concentration curve and the respiratory rate curve in sequence from top to bottom. The latest measured heart rate (shown as “80” in FIG. 8), i.e. the real-time value of the ECG parameter, is correspondingly shown on the right side of the ECG curve; the latest measured blood oxygen concentration (shown as “90” in FIG. 8), i.e. the blood oxygen parameter value, is correspondingly shown on the right side of the blood oxygen concentration curve; and the latest measured respiratory rate value (shown as “28” in FIG. 8), i.e. the respiratory rate parameter value, is correspondingly shown on the right side of the respiratory rate curve.

As shown in FIG. 8, the arterial pressure (shown as “120/80(93)” in FIG. 8), i.e. the blood pressure parameter value, is also shown below the heart rate value, blood oxygen concentration value, and respiratory rate value.

Here, historical monitoring data of a plurality of parameter values are also shown below the ECG curve, the blood oxygen concentration curve and the respiratory rate curve in the second area A1. For example, the historical monitoring data of the blood oxygen concentration value, respiratory rate value and blood pressure value measured every half hour is schematically shown in the figure.

Here, the parameter interface T2 shown in FIG. 8 is only exemplary, and the number and position of the patient status recovery parameter values displayed in the parameter interface T2 can be adjusted as required.

Pairing is performed with the portable monitoring apparatus 200 if the user chooses yes, and the communication connection with the portable monitoring apparatus 200 is established after successful pairing.

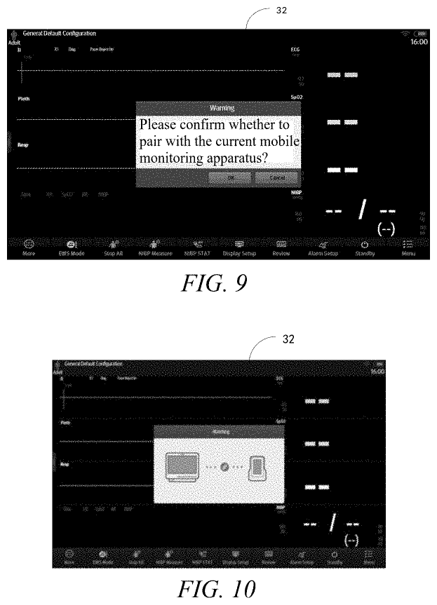

Referring to FIG. 9, provided is a schematic diagram of a pairing prompt message displayed by the monitoring device 300. As shown in FIG. 10, the processor 33 is further configured to receive a pairing request sent by the portable monitoring apparatus 200 while the communication unit 31 establishes the communication connection with the portable monitoring apparatus 200 for the first time, generate a prompt message according to the pairing request to prompt the user to choose whether to agree to pair with the portable monitoring apparatus 200, and perform a pairing operation with the portable monitoring apparatus 200 if the user chooses “yes”. Here, the pairing prompt message can be a text message, where the processor 33 can control the display screen 32 to display the pairing prompt message in text form, such as “Please confirm whether to pair with the current portable monitoring apparatus”.

Referring also to FIG. 10, provided is a schematic diagram of pairing displayed by the monitoring device 300. When the user chooses “yes”, for example, an “ok” option in FIG. 9, the processor 33 performs the pairing operation with the portable monitoring apparatus 200, and controls the display screen 32 to display a message indicating a current pairing state, for example, the message as shown in FIG. 10 indicating that the portable monitoring apparatus 200 and the target monitoring device 300 are pairing.

As mentioned above, after the monitoring device 300 is successfully paired with the portable monitoring apparatus 200, the processor 33 receives the real-time data and historical data of the patient status recovery parameter values monitored by the portable monitoring apparatus 100, and displays the parameter interface T2 including the received real-time data and historical data of the patient status recovery parameter values.

Here, when the monitoring device 300 is a bedside monitoring device 301 placed in a ward, the processor 33 is further configured to save pairing information of the portable monitoring apparatus after the monitoring device 300 is successfully paired with the portable monitoring apparatus 200, for automatic pairing later once the portable monitoring apparatus 200 is located within a preset range of the monitoring device 300, so as to establish the communication connection automatically.

In some embodiments, the processor 33 is further configured to send the pairing information of the portable monitoring apparatus 200 through the communication unit 32 to the department-level workstation device 302 and/or the hospital-level data center/hospital-level emergency center management device 303, so as to complete the pairing of the portable monitoring apparatus 200 with the department-level workstation device 302 and/or the hospital-level data center/hospital-level emergency center management device 303.

Therefore, when paired with the bedside monitoring device 301 for the first time, the portable monitoring apparatus 200 will also be paired with the department-level workstation device 302 and/or the hospital-level data center/hospital-level emergency center management device 303 through the bedside monitoring device 301. When moved out of the ward, the portable monitoring apparatus 200 can establish a communication connection with the department-level workstation device 302 and/or the hospital-level data center/hospital-level emergency center management device 303 through a WIFI network, etc.

In some embodiments, the monitoring device 300 has a mobile monitoring mode and a conventional mode, and the display screen 32 displays a main interface in the conventional mode. After the monitoring device 300 is successfully paired with the portable monitoring apparatus 200, the processor 33 is further configured to control the monitoring device 300 to switch to the mobile monitoring mode in response to an operation of a target key in the main interface of the monitoring device 300, and control the display screen 32 to display the parameter interface T2 including the received relevant data of the patient status recovery parameter values.

That is, in some embodiments, the processor 33 controls the display screen 32 to display the parameter interface T2 including relevant data of the received patient status recovery parameter values only in response to an operation of the target key on the main interface of the monitoring device 300. After the monitoring device 300 is successfully paired with the portable monitoring apparatus 200, the monitoring device 300 may only receive the real-time data and historical data of the patient status recovery parameter values monitored by the portable monitoring apparatus 200, and the display screen 32 may not display the parameter interface with the patient status recovery parameter values. When doctors or nurses need to check, they can control the display screen 32 to start displaying the parameter interface T2 by operating the target key.

Referring back to FIG. 8, the target key can be the ERAS dashboard key K0 shown at the bottom of FIG. 8.

As shown in FIG. 8, a plurality of main control keys K1 are shown on the bottom of the display screen 32 of the monitoring device 300 for controlling the monitoring device 300 to enter corresponding function modes and controlling the display screen 32 to display corresponding function interfaces.

The above-mentioned keys may all be virtual keys. In other embodiments, the above-mentioned keys may also be mechanical keys.

In some embodiments, as mentioned above, when the processor 23 of the portable monitoring apparatus 200 sends the patient status recovery parameter values to the target monitoring device 300 through the communication unit, it can also send display mode information to the target monitoring device 300 at the same time.

The processor 33 is also configured to automatically control to display the parameter interface T2 according to the display mode information when the monitoring device 300 receives the patient status recovery parameter values.

That is, in some embodiments, after the monitoring device 300 is successfully paired with the portable monitoring apparatus 200, the monitoring device 300 receives the display mode information in addition to the real-time data and historical data of the patient status recovery parameter values monitored by the portable monitoring apparatus 200. The processor 33 controls the monitoring device 300 to automatically enter the mobile monitoring mode according to the display mode information, and controls the display screen 32 to display the parameter interface T2 including the received relevant data of the patient status recovery parameter values.

Therefore, after the monitoring device 300 and the portable monitoring apparatus 200 are successfully paired, the parameter interface T2 is automatically displayed, which is convenient for viewing the relevant data of the patient status recovery parameter values through the monitoring device 300.

As mentioned above, the display mode information defines display positions of relevant data of the physiological parameter value and non-physiological parameter value in the parameter interface T2 for displaying the patient status recovery parameter values, wherein the relevant data of the physiological parameter value is displayed in the first area A1 of the parameter interface T2 and the relevant data of the non-physiological parameter value is displayed in the second area A2 of the parameter interface T2.

Therefore, when the monitoring device 300 receives the patient status recovery parameter values, it will display the parameter interface T2 according to the display mode information, and display the relevant data of physiological parameter value in the first area A1 of the parameter interface T2, and the relevant data of non-physiological parameter value in the second area A2 of the parameter interface T2.

Here, the communication unit 31 of the monitoring device 300 also includes at least one of a Bluetooth module, a WMTS communication module, and an NFC communication module. The monitoring device 300 establishes a Bluetooth connection, a WMTS communication connection, or an NFC communication connection with the portable monitoring apparatus 200 through the communication unit 31.

The communication unit 31 of the monitoring device 300 further includes a WIFI module. When the monitoring device 300 is the bedside monitoring device 301, a WIFI communication connection is also established with the department-level workstation device 302 and/or the hospital-level data center/hospital-level emergency center management device 303 through the WIFI module, and the relevant data of patient status recovery parameter values sent by portable monitoring apparatus 200 is received by the department-level workstation device 302 and/or hospital-level data center/hospital-level emergency center management device 303, wherein the relevant data of the patient status recovery parameter values is sent by the portable monitoring apparatus 200 outside a ward to the department-level workstation device 302 and/or the hospital-level data center/hospital-level emergency center management device 303 through the WIFI communication connection.

Therefore, when the patient wearing the portable monitoring apparatus 200 is outside the ward, the portable monitoring apparatus 200 can send the relevant data of the patient status recovery parameter values to the department-level workstation device 302 and/or the hospital-level data center/hospital-level emergency center management device 303 through the WIFI communication connection, which data may be then forwarded to the bedside monitoring device 301 paired with the portable monitoring apparatus 200 by the department-level workstation device 302 and/or the hospital-level data center/hospital-level emergency center management device 303, thus realizing data synchronization between multiple devices.

In some embodiments, the communication unit 31 further includes a connection interface for connecting to corresponding sensor accessories, and the processor 33 is further configured to receive the relevant data of the patient status recovery parameter values from the sensor accessories through the connection interface. The processor 33 further controls the display screen 32 to simultaneously display the relevant data of the patient status recovery parameter values received from the portable monitoring apparatus 200 and the relevant data of the patient status recovery parameter values received from the sensor accessories. The connection interface may be an accessory interface matching the sensor accessories.

That is, in some embodiments, the parameter interface T2 displayed on the display screen 32 controlled by the processor 33 include both the relevant data of the patient status recovery parameter values received by the portable monitoring apparatus 200 and the relevant data of the patient status recovery parameter values received from the sensor accessories.

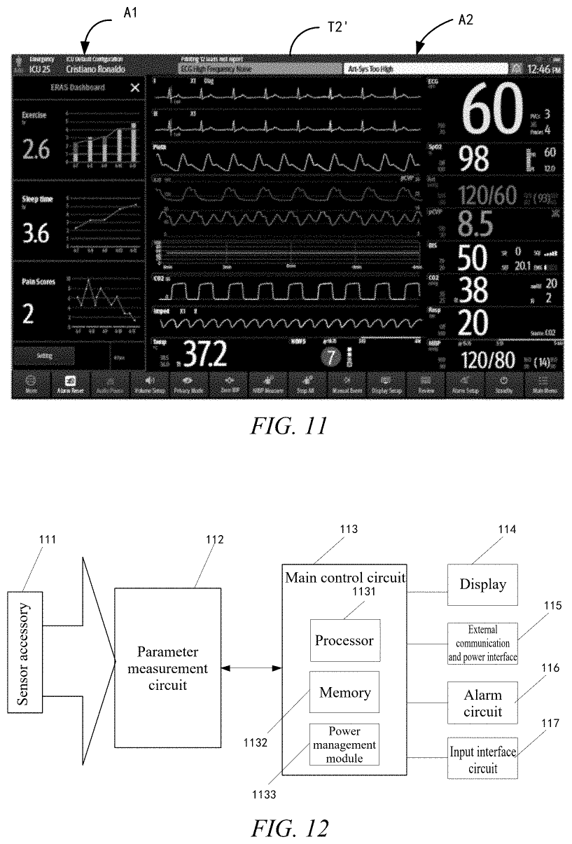

Referring also to FIG. 11, provided is a schematic diagram of a parameter interface T2′ displayed by the monitoring device 300 according to another embodiment. As shown in FIG. 11, compared with the parameter interface T2′ shown in FIG. 8, the parameter interface T2′ shown in FIG. 11 also displays the relevant data of the patient status recovery parameter values received from the sensor accessories, for example, the blood pressure parameter value of venous pressure (shown as “8.5” in FIG. 11).

In some embodiments, when the relevant data of patient status recovery parameter values received from the sensor accessories through the connection interface and the relevant data of patient status recovery parameter values received from the portable monitoring apparatus 200 have a common type of parameter values, it is also controlled to simultaneously display the relevant data of the common type of patient status recovery parameter value received from both the sensor accessory and the portable monitoring apparatus 200 in the parameter interface T2′.

For example, as shown in FIG. 11, the parameter interface T2′ also displays the arterial pressure data “120/60” received from the portable monitoring apparatus 200 and the arterial pressure data “120/80” received from the sensor accessory, simultaneously.

In some embodiments, data of the pain parameter value includes a pain level. As shown in FIG. 7, the monitoring device 300 further includes an input unit 34. The processor 33 is further configured to set the pain level in response to a pain level setting operation input through the input unit 34 to obtain the pain parameter value.

That is, in some embodiments, the pain parameter value may be generated by an input from doctors, nurses and even the patients themselves through the input unit 34 of the monitoring device 300.

The input unit 34 can be a touch panel and integrated with the display screen 32 into a touch screen. Alternatively, the input unit 34 may be a mechanical key.

Here, as shown in FIG. 11, below the historical monitoring data of a plurality of parameter values, the temperature parameter value of the last measurement (shown as “37.2° C.” in FIG. 11) is also displayed.

Therefore, the monitoring system 100 in this application can meet various monitoring requirements by monitoring the physiological parameter values and non-physiological parameter values of patients. In addition, monitoring by the portable monitoring apparatus 200 allows patients to move outside the ward, so that activity needs of the patients are satisfied as well.

The portable monitoring apparatus 200 and bedside monitoring device 301 can both be multi-parameter monitors, and the structure of the multi-parameter monitors can be seen in the following multi-parameter monitor or module assembly structure of FIG. 12.

Referring to FIG. 12, provided is a system frame diagram of a multi-parameter monitor or module assembly. The multi-parameter monitor or module assembly at least includes a parameter measurement circuit 112. The parameter measurement circuit 112 includes at least one parameter measurement circuit corresponding to a physiological parameter, including at least one of an ECG signal parameter measurement circuit, a respiratory parameter measurement circuit, a body temperature parameter measurement circuit, a blood oxygen parameter measurement circuit, a non-invasive blood pressure parameter measurement circuit, an invasive blood pressure parameter measurement circuit, etc. Each parameter measurement circuit is connected to one of sensor accessories 111 externally inserted through respective sensor interfaces. The sensor accessories 111 include corresponding detection accessories for the detection of a physiological parameter such as ECG, respiration, blood oxygen, blood pressure, and body temperature. The parameter measurement circuit 112 is mainly configured to be connected to the sensor accessories 111 to obtain the acquired physiological parameter signals, and can include at least two of the above physiological parameter measurement circuits, which can be but not limited to physiological parameter measurement circuits (modules), human physiological parameter measurement circuits (modules) or sensors acquiring human physiological parameter values, etc. Specifically, the parameter measurement circuit obtains a sampled physiological signal related to the patient from an external physiological parameter sensor accessory through an expansion interface, and processes the sampled physiological signal to obtain physiological data for alarming and displaying. The expansion interface can also be configured to output a control signal on how to obtain physiological parameter values, outputted by a main control circuit, to the external physiological parameter monitoring accessory through the corresponding interface, so as to realize the monitoring control of physiological parameter values of the patient.

For the portable monitoring apparatus 200, the parameter measurement circuit 112 can be the ECG module 203 described above. These sensor accessories 111 are sensor accessories including the aforementioned first type of sensor 21 and second type of sensor 21. For the bedside monitoring device 301, the sensor accessories 111 are external sensor accessories that can be inserted through sensor interfaces.

The multi-parameter monitor or module assembly may also include a main control circuit 113. The main control circuit 113 needs to include at least one processor 1131 and at least one memory 1132. Of course, the main control circuit may also include at least one of a power management module 1133, a power IP module, an interface conversion circuit, etc. The power management module is configured to control the power on and off of an entire machine, a power-on sequence of each power domain inside a board card, and battery charging and discharging. The power IP module is a separate power module firmed by associating a principle diagram of a power supply circuit unit that is frequently and repeatedly invoked, with a PCB layout. That is, an input voltage is converted into an output voltage through a predetermined circuit, wherein the input voltage and the output voltage are different. For example, the voltage of 15 V is converted into 1.8 V, 3.3 V, 3.8 V, etc. It can be understood that the power IP module may be single-channel or multi-channel. When the power IP module is single-channel, the power IP module can convert one input voltage into one output voltage. When the power IP module is multi-channel, the power IP module can convert one input voltage into a plurality of output voltages, and voltage values of the plurality of output voltages may be the same or different, such that different voltage requirements of a plurality of electronic components can be met at the same time. In addition, the module has few external interfaces, and works in the system as a black box decoupled from an external hardware system, which improves the reliability of the entire power system. The interface conversion circuit is configured to convert a signal outputted by a main control minimum system module (i.e., at least one processor and at least one memory in the main control circuit) into an input standard signal required to be received by an actual external device. For example, supporting an external VGA display function is to convert an RGB digital signal outputted by a main control CPU into a VGA analog signal, and supporting an external network function is to convert an RMII signal into a standard network differential signal.

In addition, the multi-parameter monitor or module assembly may also include one or more of a local display 114, an alarm circuit 116, an input interface circuit 117, and an external communication and power interface 115. The main control circuit is configured to coordinate and control board cards, circuits and devices in the multi-parameter monitor or module assembly. In this embodiment, the main control circuit is configured to control data interaction and transmission of control signals between the parameter measurement circuit 112 and the communication interface circuit and to transmit the physiological data to the display 114 for display, and can also receive user control instructions input from a touch screen or physical input interface circuits such as keyboards and keys, and of course, can also output control signals on how to acquire the physiological parameter values. The alarm circuit 116 may be an audible and visual alarm circuit. The main control circuit completes the calculation of physiological parameter values, and sends the calculation results and waveforms of the parameter values to a host (such as a host computer with a display, a PC, a central station, etc.) through the external communication and power interface 115. The external communication and power interface 115 can be one of or a combination of Ethernet, Token Ring, Token Bus, and a local area network interface composed of fiber distributed data interface (FDDI), which is the backbone of these three networks, or can also be one of or a combination of wireless interfaces such as infrared, Bluetooth, WIFI and WMTS communication interfaces, or can also be one of or a combination of wired data connection interfaces such as RS232 and USB interfaces. The external communication and power interface 115 may also be one of a wireless data transmission interface and a wired data transmission interface or a combination thereof. The host may be any computing device, such as a main unit of the monitor, an electrocardiograph machine, an ultrasonic diagnosis instrument, and a computer, and can form the monitoring device once installed with matching software. The host may alternatively be a communication device such as a mobile phone, and the multi-parameter monitor or module assembly sends, by using a Bluetooth interface, data to the mobile phone supporting Bluetooth communication, so as to implement remote transmission of data.

For the portable monitoring apparatus 200, the local display 114 is the display screen 24, the input interface circuit 117 is the input unit 26, and the external communication and power interface 115 can be the aforementioned communication unit 25. For the bedside monitoring device 200, the local display 114 is the display screen 32, the input interface circuit 117 is the input unit 34, and the external communication and power interface 115 can be the aforementioned communication unit 31.

The multi-parameter monitoring module assembly may be arranged outside a housing of the monitor as an independent externally inserted parameter module, may be inserted into the main unit (including a main control board) of the monitor to form a plug-in monitor to serve as a part of the monitor, or may be connected to the main unit (including the main control board) of the monitor through a cable, and the externally inserted parameter module is used as an external accessory of the monitor. Certainly, the parameter processing module may also be built in the housing to be integrated with a main control module, or physically separated and arranged in the housing to form an integrated monitor.

Here, as shown in FIG. 2, the portable monitoring apparatus 200 further includes a memory 27. The memory 27 can be configured to store the aforementioned relevant data of the patient status recovery parameter values. As shown in FIG. 7, the monitoring device 300 also includes a memory 35. The memory 35 can be configured to store relevant data of the patient status recovery parameter values received by the monitoring device 300.

In some embodiments, the memory 27 further stores program instructions for the processor 23 of the portable monitoring apparatus 200 to invoke for execution of the aforementioned functions. The memory 35 of the monitoring device 300 also stores program instructions for the processor 33 of the monitoring device 300 to invoke for execution of the aforementioned functions.

The memories 27 and 35 may include a high-speed random access memory, and may alternatively include a nonvolatile memory, such as a hard disk, internal memory, plug-in hard disk, smart media card (SMC), secure digital (SD) card, flash card, multiple disk memory devices, and flash memory device, or other volatile solid-state memory devices.

The processors 23 and 33 may be a central processing unit (CPU), or may be other general-purpose processors, a digital signal processor (DSP), an application specific integrated circuit (ASIC), a field-programmable gate array (FPGA), or other programmable logic devices, discrete gates or transistor logic devices, discrete hardware components, etc. The general-purpose processor may be a microprocessor, or the processor may be any conventional processor, etc.

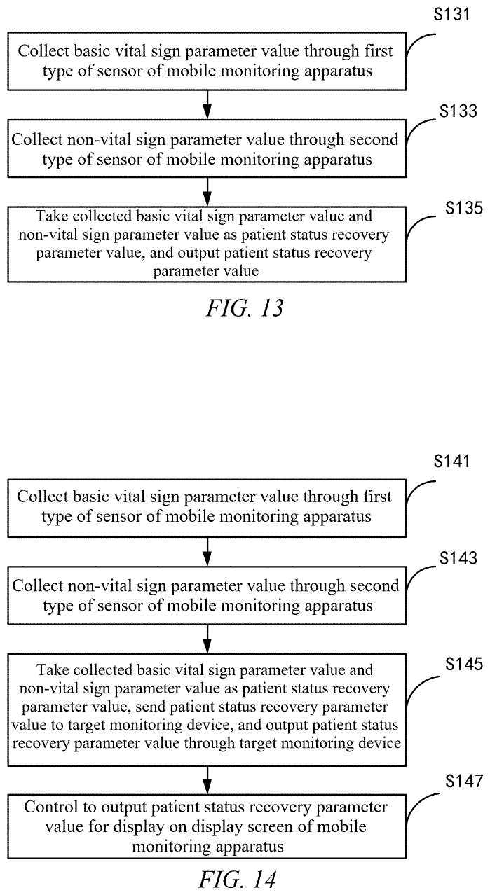

Referring to FIG. 13, provided is a flow chart of a patient status monitoring method according to an embodiment. The patient status monitoring method can be applied to the above portable monitoring apparatus. As shown in FIG. 13, the method includes the following steps:

S131: acquiring, by a first type of sensor of the portable monitoring apparatus, a physiological parameter value, wherein the physiological parameter value includes at least one of an ECG parameter value, a respiratory parameter value, a blood oxygen parameter value, a blood pressure parameter value, and a body temperature parameter value;

S133: acquiring, by a second type of sensor of the portable monitoring apparatus, a non-physiological parameter value, wherein the non-physiological parameter value includes at least one of a sleep parameter value, a motion parameter value, and a pain parameter value; and

S135: taking the acquired physiological parameter value and non-physiological parameter value as patient status recovery parameter values, and outputting the patient status recovery parameter values, wherein the acquired physiological parameter value and non-physiological parameter value are summarized to form patient status recovery parameter values.

Here, steps S131 and S133 may be performed simultaneously or sequentially.

In some embodiments, the portable monitoring apparatus further includes a display screen, and the above-mentioned “outputting the patient status recovery parameter values” includes: controlling to display the patient status recovery parameter values on the display screen of the portable monitoring apparatus.

That is, the patient status recovery parameter values are displayed on the display screen of the portable monitoring apparatus.

Further, the above-mentioned “controlling to display the patient status recovery parameter values on the display screen of the portable monitoring apparatus” includes: controlling the display screen to display a parameter interface including at least one parameter value of the patient status recovery parameter values; and controlling to switch the parameter value displayed in the parameter interface in response to an operation of a user or according to a preset time interval.

Therefore, when the patient status recovery parameter values include several parameter values, and only some of the parameter values are displayed in the parameter interface at one time, the parameter values displayed in the parameter interface can be controlled to switch in response to an operation by the user or according to a preset time interval, for example, switching the blood pressure parameter value currently displayed in the parameter interface to the body temperature parameter value for display, etc.

The controlling to display the patient status recovery parameter values on the display screen of the portable monitoring apparatus includes: when the patient status recovery parameter value displayed on the display screen is the motion parameter value, controlling to display sub-motion parameter values included in the motion parameter value in a ring-shaped form, the sub-motion parameter value including at least one of a number of steps, a step frequency, a distance of motion, calories, a time of motion and a route of motion.

Here, the portable monitoring apparatus further includes a communication unit, and the above-mentioned “outputting the patient status recovery parameter values” may further include: establishing a communication connection with the target monitoring device through the communication unit; and sending the patient status recovery parameter values to the target monitoring device, and outputting the patient status recovery parameter values through the target monitoring device, wherein the target monitoring device includes at least one of a bedside monitoring device, a department-level workstation device and a hospital-level data center/hospital-level emergency center management device.

In some embodiments, the above-mentioned “sending the patient status recovery parameter values to the target monitoring device and outputting the patient status recovery parameter values through the target monitoring device” includes: while sending the patient status recovery parameter values to the target monitoring device, also sending display mode information to the target monitoring device, such that the target monitoring device displays the patient status recovery parameter values according to the display mode information.

Here, the display mode information defines display positions of relevant data of the physiological parameter value and non-physiological parameter value in the display interface for displaying the patient status recovery parameter values, wherein the relevant data of the physiological parameter value is displayed in a first display area of the display interface and the relevant data of the non-physiological parameter value is displayed in a second display area of the display interface.

Here, the relevant data of the physiological parameter value includes real-time data of the physiological parameter value, and the relevant data of the non-physiological parameter value includes real-time data of the non-physiological parameter value.

Here, the relevant data of the non-physiological parameter value further includes trend data of the non-physiological parameter value. In some embodiments, the trend data includes trend data of the plurality of non-physiological parameter values detected within the last week.

Here, when the target monitoring device is the bedside monitoring device, the method further includes: performing a pairing operation with the target monitoring device while the communication unit establishes the communication connection between the portable monitoring apparatus and the target monitoring device for the first time; and establishing the communication connection between the portable monitoring apparatus and the target monitoring device after successful pairing with the target monitoring device.

In some embodiments, before “performing the pairing operation with the target monitoring device”, the method further includes: generating a pairing prompt message to prompt the user to choose whether to pair the portable monitoring apparatus with the target monitoring device.

The above-mentioned “performing the pairing operation with the target monitoring device” includes: performing the pairing operation with the target monitoring device if the user chooses yes.

Further, the above-mentioned “sending the patient status recovery parameter values to the target monitoring device” includes: after the portable monitoring apparatus is successfully paired with the target monitoring device, controlling to synchronize real-time data and historical data of the patient status recovery parameter values detected by the portable monitoring apparatus to the target monitoring device.

Here, the communication unit of the portable monitoring apparatus includes and a WIFI communication module and at least one of a Bluetooth module, a WMTS communication module and an NFC communication module. When the portable monitoring apparatus is located in a ward, the portable monitoring apparatus establishes a Bluetooth connection, a WMTS communication connection or an NFC communication connection with the bedside monitoring device through the Bluetooth module, the WMTS communication module or the NFC communication module. The above-mentioned “controlling to synchronize real-time data and historical data of the patient status recovery parameter values detected by the portable monitoring apparatus to the target monitoring device” includes: controlling to synchronize the real-time data and historical data of the patient status recovery parameter values detected by the portable monitoring apparatus to the bedside monitoring device through the Bluetooth connection, WMTS communication connection or NFC communication connection.

Here, when the portable monitoring apparatus is located outside the ward, the portable monitoring apparatus establishes a WIFI communication connection with the department-level workstation device and/or the hospital-level data center/hospital-level emergency center management device through the WIFI module. The above-mentioned “controlling to synchronize real-time data and historical data of the patient status recovery parameter values detected by the portable monitoring apparatus to the target monitoring device” includes: controlling to synchronize the real-time data and historical data of the patient status recovery parameter values detected by the portable monitoring apparatus to the department-level workstation device and/or the hospital-level data center/hospital-level emergency center management device through the WIFI communication connection, and then further synchronizing the real-time data and historical data of the patient status recovery parameter values to the bedside monitoring device through the department-level workstation device and/or the hospital-level data center/hospital-level emergency center management device.

In some embodiments, the above method further includes: controlling the display screen to display a lock screen interface when the portable monitoring apparatus has received no user operation for a time longer than a preset time duration; and resuming the display of the parameter interface in response to any sliding operation by the user on the display screen.

In some embodiments, the portable monitoring apparatus further includes an input unit, and the method further includes: setting a current pain level of the user of the portable monitoring apparatus in response to a pain level setting operation input through the input unit.

Referring to FIG. 14, provided is a flow chart of a patient status monitoring method according to another embodiment. The patient status monitoring method can be applied to the above portable monitoring apparatus 200. The portable monitoring apparatus 200 includes a communication unit and a display screen. As shown in FIG. 14, the method includes the following steps:

S141: acquiring, by a first type of sensor of the portable monitoring apparatus, a physiological parameter value, wherein the physiological parameter value includes at least one of an ECG parameter value, a respiratory parameter value, a blood oxygen parameter value, a blood pressure parameter value, and a body temperature parameter value;

S143: acquiring, by a second type of sensor of the portable monitoring apparatus, a non-physiological parameter value, wherein the non-physiological parameter value includes at least one of a sleep parameter value, a motion parameter value, and a pain parameter value;

S145: taking the acquired physiological parameter value and non-physiological parameter value as patient status recovery parameter values, sending the patient status recovery parameter values to a target monitoring device, and outputting the patient status recovery parameter values through the target monitoring device, wherein the target monitoring device includes at least one of a bedside monitoring device, a department-level workstation device and a hospital-level data center/hospital-level emergency center management device; and

S147: controlling to display the patient status recovery parameter values on the display screen of the portable monitoring apparatus.

That is, when the patient status recovery parameter values acquired by the portable monitoring apparatus are sent to the target monitoring device for output, they will also be displayed on the display screen of the portable monitoring apparatus.

Before step S145, the above method may further include: establishing a communication connection with the target monitoring device through the communication unit.

Here, please refer to the description of FIG. 13 for the description of the related steps of the patient status monitoring method shown in FIG. 14 and the patient status monitoring method shown in FIG. 13. The additional steps related to FIG. 13 can also be applied to the patient status monitoring method shown in FIG. 14.

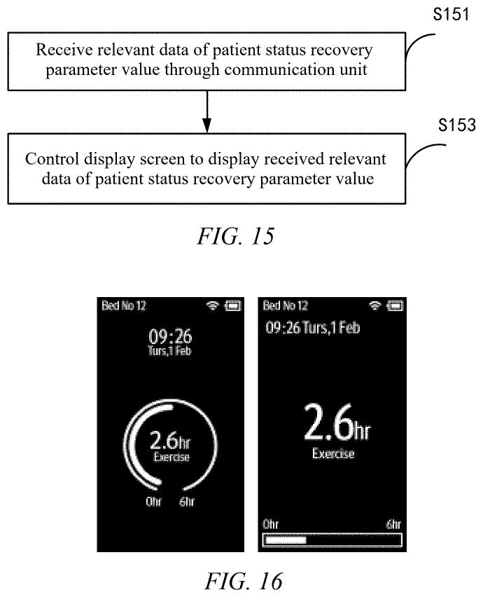

Referring to FIG. 15, provided is a flow chart of a patient status monitoring method according to a further embodiment. The patient status monitoring method can be applied to the above monitoring device 300. The monitoring device 300 includes a communication unit and a display screen. As shown in FIG. 15, the method includes the following steps:

S151: receiving relevant data of patient status recovery parameter values through the communication unit; and

S153: controlling the display screen to display the received relevant data of the patient status recovery parameter values, wherein the patient status recovery parameter values include a physiological parameter value and a non-physiological parameter value, the physiological parameter value includes at least one of an ECG parameter value, a respiratory parameter value, a blood oxygen parameter value, a blood pressure parameter value, and a body temperature parameter value, and the non-physiological parameter value includes at least one of a sleep parameter value, a motion parameter value, and a pain parameter value.

Here, step S151 may specifically include: establishing, by the communication unit, a communication connection with the portable monitoring apparatus, and receiving the relevant data of the patient status recovery parameter values acquired by the portable monitoring apparatus from the portable monitoring apparatus through the communication unit.

Here, the above-mentioned “controlling the display screen to display the received relevant data of the patient status recovery parameter values” includes: controlling the display screen to display a parameter interface including the received relevant data of the patient status recovery parameter values, wherein the relevant data of the non-physiological parameter value is displayed in a first area of the parameter interface and the relevant data of the physiological parameter value is displayed in a second area of the parameter interface.

Here, the relevant data of the physiological parameter value includes real-time data of the physiological parameter value, and the relevant data of the non-physiological parameter value includes real-time data of the non-physiological parameter value.

Here, the relevant data of the non-physiological parameter value further includes trend data of the non-physiological parameter value. In some embodiments, the trend data includes trend data of the plurality of non-physiological parameter values detected within a preset time period before a current time, for example, trend data of the plurality of non-physiological parameter values detected within the last week.

In some embodiments, the method may further include: receiving a pairing request sent by the portable monitoring apparatus while the communication unit establishes the communication connection with the portable monitoring apparatus for the first time; generating a prompt message according to the pairing request to prompt a user to choose whether to agree to pair with the portable monitoring apparatus; and pairing with the portable monitoring apparatus if the user chooses yes, and establishing the communication connection with the portable monitoring apparatus after successful pairing.

Here, the method further includes: saving pairing information of the portable monitoring apparatus after the monitoring device is successfully paired with the portable monitoring apparatus, for automatic pairing later once the portable monitoring apparatus is located within a preset range of the monitoring device.

In some embodiments, the target monitoring device is a bedside monitoring device placed in a ward, and the method further includes: sending the pairing information of the portable monitoring apparatus to a department-level workstation device and/or a hospital-level data center/hospital-level emergency center management device, so as to complete the pairing of the portable monitoring apparatus with the department-level workstation device and/or the hospital-level data center/hospital-level emergency center management device.

Here, the communication unit of the monitoring device includes at least one of a Bluetooth module, a WMTS communication module, and an NFC communication module, and the communication unit establishes a Bluetooth connection, a WMTS communication connection or an NFC communication connection with the portable monitoring apparatus.

The monitoring device has a mobile monitoring mode and a conventional mode, and the display screen displays a main interface in the conventional mode, and the above-mentioned “controlling the display screen to display a parameter interface including the received relevant data of the patient status recovery parameter values” includes: controlling the monitoring device to switch to the mobile monitoring mode in response to an operation of a target key in the main interface of the monitoring device; and in the mobile monitoring mode, controlling the display screen to display a parameter interface including the received relevant data of the patient status recovery parameter values.

In some embodiments, the communication unit further includes a connection interface for connecting to corresponding sensor accessories, and the above method further includes: receiving the relevant data of the patient status recovery parameter values from the sensor accessories through the connection interface; The above-mentioned “controlling the display screen to display the received relevant data of the patient status recovery parameter values” includes: controlling the display screen to simultaneously display the relevant data of the patient status recovery parameter values received from the portable monitoring apparatus and the relevant data of the patient status recovery parameter values received from the sensor accessories.

Here, the communication unit includes a WIFI module, and the above-mentioned “receiving the relevant data of the patient status recovery parameter values acquired by the portable monitoring apparatus from the portable monitoring apparatus through the communication unit” further includes:

establishing a WIFI communication connection with a department-level workstation device and/or a hospital-level data center/hospital-level emergency center management device through the WIFI module, and receiving the relevant data of the patient status recovery parameter values sent by the portable monitoring apparatus through the department-level workstation device and/or the hospital-level data center/hospital-level emergency center management device, wherein the relevant data of the patient status recovery parameter values is sent by the portable monitoring apparatus outside a ward to the department-level workstation device and/or the hospital-level data center/hospital-level emergency center management device through the WIFI communication connection.

In some embodiments, data of the pain parameter value includes a pain level, the monitoring device further includes an input unit, and the method further includes: setting the pain level in response to a pain level setting operation input through the input unit to obtain the data of the pain parameter value.

Here, the patient status monitoring methods in various embodiments of this application correspond to the aforementioned monitoring system 100, and the related steps and the functional operations performed by the monitoring system 100 can be referred to each other correspondingly, which will not be described in further detail here.

In some embodiments, this application also provides a computer-readable storage medium. The aforementioned computer-readable storage medium stores a plurality of program instructions for the processor 23 or the processor 33 to invoke and execute.

After the plurality of program instructions stored in the computer-readable storage medium are invoked and executed by the processor 23 of the portable monitoring apparatus 200, part or all of the steps or any combination of the steps in the method shown in either of FIGS. 13 and 14 can be performed. After the plurality of program instructions stored in the computer-readable storage medium are invoked and executed by the processor 33 of the monitoring device 300, part or all of the steps or any combination of the steps in the method shown in FIG. 15 can be performed.

Therefore, according to this application, the physiological parameter value and the non-physiological parameter value may be obtained at the same time, and physiological status such as ECG and respiration as well as non-physiological status such as sleep and motion states of the user may be obtained, so that the status of the patient may be detected more comprehensively. In addition, as the portable monitoring apparatus 200 may be a wearable monitoring apparatus, which is configured to be worn by a patient for activities outside the ward, and at the same time can continuously monitor the patient status recovery parameter value, the need to monitor status of the patient (for example, a sub-severe patient) during his/her activities outside the ward is satisfied.

Specifically, referring to FIG. 16, provided is a schematic diagram of an interface displayed on a display screen of a portable monitoring apparatus according to an embodiment of the disclosure. The processor 23 controls the display screen to display an amount of motion of the patient, for example, 2.6 hours of motion, and controls to display the total number of hours the patient needs to motion every day, for example, 6 hours. Further, in one embodiment, the processor 23 controls the display screen to display a progress bar including the amount of motion and the total number of motion hours required, so that the patient can be conspicuously reminded to have activities corresponding to the required amount of motion.

Further, after the wearable piece 201 is paired with the bedside monitoring device 301 (as shown in FIG. 1), or after the wearable piece 201 is paired with the bedside monitoring device 301 and the bedside monitoring device 301 establishes a communication connection with the department-level workstation device 302 (as shown in FIG. 1), synchronous display can be realized between the wearable piece 201 and the bedside monitoring device 301 and/or the department-level workstation device 302.

An embodiment of the disclosure also provides a wearable piece including a processor and a motion sensor, wherein the processor is electrically connected to the motion sensor, and the processor acquires a motion parameter value of a patient wearing the wearable piece according to a motion sensing signal generated by the motion sensor and deduces an motion amount and/or sleep status of the patient wearing the wearable piece from the motion parameter value.

In some embodiments, data processing can be carried out in combination with the motion parameter value obtained by the motion sensor during data processing by the processor based on at least one of the acquired ECG parameter value, respiratory parameter value, blood oxygen parameter value, blood pressure parameter value, and body temperature parameter value, so as to eliminate the interference with at least one of the ECG parameter value, respiratory parameter value, blood oxygen parameter value, blood pressure parameter value, and body temperature parameter value due to movement of the patient wearing the wearable piece.

The description has been made with reference to various exemplary embodiments herein. However, those skilled in the art would have appreciated that changes and modifications could have been made to the exemplary embodiments without departing from the scope herein. For example, various operation steps and assemblies for executing operation steps may be implemented in different ways according to a specific application or considering any number of cost functions associated with the operation of the system (for example, one or more steps may be deleted, modified or incorporated into other steps).

In addition, as understood by those skilled in the art, the principles herein may be reflected in a computer program product on a computer-readable storage medium that is pre-installed with computer-readable program codes. Any tangible, non-transitory computer-readable storage medium can be used, including magnetic storage devices (hard disks, floppy disks, etc.), optical storage devices (CD-ROM, DVD, Blu Ray disks, etc.), flash memories, and/or the like. These computer program instructions can be loaded onto a general-purpose computer, a dedicated computer, or other programmable data processing device to form a machine, such that these instructions executed on a computer or other programmable data processing apparatus can generate an apparatus that implements a specified function. These computer program instructions can also be stored in a computer-readable memory that can instruct a computer or other programmable data processing device to operate in a specific manner, such that the instructions stored in the computer-readable memory can form a manufactured product, including an implementation apparatus that implements a specified function. The computer program instructions can also be loaded onto a computer or other programmable data processing device, such that a series of operating steps are executed on the computer or other programmable device to produce a computer-implemented process, such that the instructions executed on the computer or other programmable device can provide steps for implementing a specified function.

Although the principles herein have been shown in various embodiments, many modifications of structures, arrangements, ratios, elements, materials, and components that are particularly suitable for specific environments and operating requirements can be made without departing from the principles and scope of the disclosure. The above modifications and other changes or amendments will be included within the scope herein.

The above specific description has been described with reference to various embodiments. However, those skilled in the art would have appreciated that various modifications and changes could have been made without departing from the scope of the disclosure. Therefore, consideration of the disclosure will be in an illustrative rather than a restrictive sense, and all such modifications will be included within the scope thereof. Also, the advantages, other advantages and the solutions to problems related to various embodiments have been described above. However, the benefits, advantages, solutions to problems, and any elements that can produce these, or solutions that make them more explicit, should not be interpreted as critical, necessary, or essential. The term “comprise”, “include”, and any other variants thereof used herein are non-exclusive, so that the process, method, document, or device that includes a list of elements includes not only these elements, but also other elements that are not explicitly listed or do not belong to the process, method, system, document, or device. Furthermore, the term “coupling” and any other variations thereof used herein refer to physical connection, electrical connection, magnetic connection, optical connection, communication connection, functional connection, and/or any other connection.

Those skilled in the art will recognize that many changes can be made to the details of the above-described embodiments without departing from the basic principles of the disclosure. Therefore, the scope of the disclosure should be determined only by the claims as follows.

1. A portable monitoring apparatus, comprising:
a first type of sensor configured to acquire a physiological parameter value, which comprises at least one of an electrocardiogram (ECG) parameter value, a respiratory parameter value, a blood oxygen parameter value, a blood pressure parameter value, and a body temperature parameter value;
a second type of sensor configured to acquire a non-physiological parameter value, which comprises at least one of a sleep parameter value, a motion parameter value, and a pain parameter value; and
a processor configured to take the acquired physiological parameter value and non-physiological parameter value as patient status recovery parameter values and output the patient status recovery parameter values.

2. The portable monitoring apparatus of claim 1, further comprising a display screen, wherein the processor is further configured to control the display screen to display the patient status recovery parameter values.

3. The portable monitoring apparatus of claim 2, wherein the processor is further configured to control the display screen to display a parameter interface comprising at least one parameter value of the patient status recovery parameter values, and to switch the parameter value displayed on the parameter interface in response to an operation of a user or according to a preset time interval.

4. The portable monitoring apparatus of claim 2 or 3, wherein, when the patient status recovery parameter value displayed on the display screen is the motion parameter value, the processor is further configured to control the display screen to display a sub-motion parameter value included in the motion parameter value, and wherein the sub-motion parameter value comprises at least one of a number of steps, a step frequency, a distance of motion, calories, a time of motion and a path of motion.

5. The portable monitoring apparatus of claim 3, wherein the processor is further configured to control the display screen to display a lock screen interface when the portable monitoring apparatus receives no user operation for a time longer than a preset time duration, and to resume the display screen to display the parameter interface in response to any sliding operation or tap operation by the user on the display screen for unlocking the display screen.

6. The portable monitoring apparatus of claim 5, wherein a number of types of patient recovery parameter values included in the lock screen interface displayed by the portable monitoring apparatus is less than a number of types of patient recovery parameter values included in the parameter interface displayed after the user unlocks the display screen.

7. The portable monitoring apparatus of claim 5, wherein the lock screen interface is a simplified interface of the parameter interface when the display screen is unlocked.

8. The portable monitoring apparatus of claim 1, further comprising a communication unit, wherein the processor is further configured to establish a communication connection between the portable monitoring apparatus and a target monitoring device through the communication unit and to send the patient status recovery parameter values to the target monitoring device through the communication unit, and the patient status recovery parameter values are outputted through the target monitoring device; and wherein the target monitoring device comprises at least one of a bedside monitoring device, a department-level workstation device and a hospital-level data center/hospital-level emergency center management device.

9. The portable monitoring apparatus of claim 8, wherein, while sending the patient status recovery parameter values to the target monitoring device through the communication unit, the processor is further configured to send display mode information to the target monitoring device, such that the target monitoring device displays a corresponding parameter interface according to the display mode information for displaying the patient status recovery parameter values.

10. The portable monitoring apparatus of claim 9, wherein the display mode information defines display positions in the parameter interface for the physiological parameter value and the non-physiological parameter value, wherein the physiological parameter value is displayed in a first area of the parameter interface and the non-physiological parameter value is displayed in a second area of the parameter interface, and wherein the physiological parameter value comprises at least real-time data of the physiological parameter value, and/or the non-physiological parameter value comprises at least real-time data of the non-physiological parameter value.

11. The portable monitoring apparatus of claim 10, wherein the non-physiological parameter value further comprises trend data of the non-physiological parameter value.

12. The portable monitoring apparatus of claim 8, wherein the processor is further configured to perform a pairing operation between the portable monitoring apparatus and the target monitoring device while the communication unit establishes the communication connection between the portable monitoring apparatus and the target monitoring device for the first time, and the communication connection between the portable monitoring apparatus and the target monitoring device is established after the portable monitoring apparatus is successfully paired with the target monitoring device.

13. The portable monitoring apparatus of claim 12, wherein, before performing the pairing operation, the processor is further configured to generate a pairing prompt message to remind a user to choose whether to pair the portable monitoring apparatus with the target monitoring device or not, and to perform the pairing operation between the portable monitoring apparatus and the target monitoring device if the user chooses to pair.

14. The portable monitoring apparatus of claim 12 or 13, wherein, after the portable monitoring apparatus is successfully paired with the target monitoring device, the processor is further configured to synchronize real-time data and historical data of the patient status recovery parameter values detected by the portable monitoring apparatus to the target monitoring device.

15. The portable monitoring apparatus of claim 14, wherein, when the target monitoring device is a bedside monitoring device, the communication unit comprises a WIFI communication module and at least one of a Bluetooth module, a WMTS communication module and an NFC communication module; when the portable monitoring apparatus is located in a ward, the portable monitoring apparatus establishes a Bluetooth connection, a WMTS communication connection or an NFC communication connection with the bedside monitoring device through the Bluetooth module, the WMTS communication module or the NFC communication module, respectively, and synchronizes the real-time data and the historical data of the patient status recovery parameter values detected by the portable monitoring apparatus to the bedside monitoring device; and when the portable monitoring apparatus is located outside a ward, the portable monitoring apparatus establishes a WIFI communication connection with the department-level workstation device and/or the hospital-level data center/hospital-level emergency center management device through the WIFI module, and synchronizes the real-time data and the historical data of the patient status recovery parameter values detected by the portable monitoring apparatus to the department-level workstation device and/or the hospital-level data center/hospital-level emergency center management device, and then the real-time data and the historical data of the patient status recovery parameter values are further synchronized to the bedside monitoring device through the department-level workstation device and/or the hospital-level data center/hospital-level emergency center management device.

16. The portable monitoring apparatus of claim 14, wherein, when the target monitoring device is a bedside monitoring device, the communication unit comprises a WMTS communication module and at least one of a Bluetooth module and an NFC communication module; when the portable monitoring apparatus is located in a ward, the portable monitoring apparatus establishes a Bluetooth connection or an NFC communication connection with the bedside monitoring device through the Bluetooth module or the NFC communication module, respectively, and synchronizes the real-time data and the historical data of the patient status recovery parameter values detected by the portable monitoring apparatus to the bedside monitoring device; and when the portable monitoring apparatus is located outside a ward, the portable monitoring apparatus establishes a WMTS communication connection with the department-level workstation device and/or the hospital-level data center/hospital-level emergency center management device through the WMTS module, and synchronizes the real-time data and the historical data of the patient status recovery parameter values detected by the portable monitoring apparatus to the department-level workstation device and/or the hospital-level data center/hospital-level emergency center management device, and then the real-time data and the historical data of the patient status recovery parameter values are further synchronized to the bedside monitoring device through the department-level workstation device and/or the hospital-level data center/hospital-level emergency center management device.

17. The portable monitoring apparatus of claim 1, further comprising an input unit, wherein the processor is further configured to set a current pain level for the portable monitoring apparatus in response to a pain level setting operation inputted by a user through the input unit, so as to obtain the pain parameter value.

18. A monitoring system, comprising a portable monitoring apparatus and a monitoring device, wherein the portable monitoring apparatus comprises:
a first type of sensor configured to acquire a physiological parameter value, which comprises at least one of an electrocardiogram (ECG) parameter value, a respiratory parameter value, a blood oxygen parameter value, a blood pressure parameter value, and a body temperature parameter value;
a second type of sensor configured to acquire a non-physiological parameter value, which comprises at least one of a sleep parameter value, a motion parameter value, and a pain parameter value;
a first communication unit; and
a first processor, wherein the first processor is configured to establish a communication connection between the portable monitoring apparatus and the monitoring device through the first communication unit and to take the acquired physiological parameter value and non-physiological parameter value as patient status recovery parameter values and send the patient status recovery parameter values to the monitoring device through the first communication unit;
and wherein the monitoring device comprises:
a second communication unit;
a second display screen; and
a second processor configured to receive the patient status recovery parameter values from the portable monitoring apparatus through the send communication unit and control the second display screen to display the received patient status recovery parameter values; and wherein the monitoring device is at least one of a bedside monitoring device, a department-level workstation device and a hospital-level data center/hospital-level emergency center management device.

19. The monitoring system of claim 18, wherein the monitoring device comprises a mobile monitoring mode and a conventional monitoring mode, the second display screen displays a main interface in the conventional monitoring mode, and the second processor is further configured to control the monitoring device to switch to the mobile monitoring mode in response to an operation of a target key in the main interface of the monitoring device, and control the second display screen to display a parameter interface comprising the received patient status recovery parameter values.

20. A monitoring device, comprising:
a communication unit;
a display screen; and
a processor configured to receive patient status recovery parameter values from a portable monitoring apparatus through the communication unit and control the display screen to display the patient status recovery parameter values; wherein the patient status recovery parameter values comprise a physiological parameter value and a non-physiological parameter value, and wherein the physiological parameter value comprises at least one of an ECG parameter value, a respiratory parameter value, a blood oxygen parameter value, a blood pressure parameter value, and a body temperature parameter value, while the non-physiological parameter value comprises at least one of a sleep parameter value, a motion parameter value, and a pain parameter value.

21-64. (canceled)
